(19) United States
(12) Patent Application Publication
(10) Pub. No.: US 2019/0090458 A1
(43) Pub. Date: Mar. 28, 2019

(54) DEVICE FOR OBTAINING AGE/SIZE-MATCHED NEMATODES AND METHODS OF USE

(71) Applicant: University of Alaska, Fairbanks, Fairbanks, AK (US)

(72) Inventors: Elena Vayndorf, Rego Park, NY (US); Skyler Hunter, North Pole, AK (US)

(73) Assignee: University of Alaska, Fairbanks, Fairbanks, AK (US)

(21) Appl. No.: 16/009,542

(22) Filed: Jun. 15, 2018

Related U.S. Application Data
(63) Continuation of application No. 62/521,066, filed on Jun. 16, 2017.

(51) Int. Cl. A01K 29/00; A01K 67/033

(52) U.S. Cl. CPC A01K 29/00; A01K 67/0336

(57) ABSTRACT

Provided herein are devices, systems and methods for separating age and size matched nematodes from a mixed size population of nematodes and/or debris. The device and system use a filter membrane with a defined pore size located between an upper housing with a first chamber adapted to receive a liquid sample comprising the mixed size population nematodes and a lower housing with a second chamber. Use of the system, along with a sample vessel, provide the user a method for efficiently and effectively isolating an age and size synchronized population of nematodes.

### CROSS-REFERENCE TO RELATED APPLICATIONS

This application claims the benefit of U.S. Provisional Patent Application No. 62/521,066, filed on 16 Jun. 2017, the contents of which are incorporated herein by reference in its entirety.

### FIELD OF THE INVENTION

This application pertains generally to a device, system, and methods for separating nematodes by size and age and/or debris from a mixed size population of nematodes.

### BACKGROUND OF THE INVENTION

The nematode roundworm, especially Caenorhabditis elegans (C. elegans), is a premier model organism. In addition to the straightforward and controlled nature of their cultivation in the laboratory, the genome of C. elegans is sequenced and the developmental fate of each cell is known. Due to these features, nematodes are a widely used model organism for genetic studies. However, along with these beneficial characteristics come some challenges for researchers. Due to their rapid generation time, nematode populations can quickly run out of food and/or become mixed populations with multiple generations and developmental stages present at once. Thus, experiments performed on solid nematode growth media (NGM) require researchers to physically move animals to fresh plates before the bacterial food source depletes and new larvae develop. This frequent transferring of animals—required to prevent the experimental populations from becoming mixed with offspring generations—can be tedious. Still, some experiments require both large numbers of animals and extended time points (e.g., DNA or RNA extraction in mid- to late-adulthood). This presents the challenge of accurately maintaining a synchronized population and transferring large numbers of animals.

Current methods of transferring nematodes cultured on NGM are: picking or washing the animals from plate to plate; chemically treating animals (e.g. the DNA replication inhibitor 5-fluorodeoxyuridine (FUDR)); or using flow cytometry to sort animals in multi-well plates. Picking involves the use of a hand tool, made with either a thin platinum wire or an eyelash, to manually transfer individual or multiple animals. This method is accurate but requires both skill and time and is a limitation for studies involving large numbers of animals. Picking also may be physically damaging and stressful to the animals by potentially subjecting individuals to unnatural and inconsistent amounts of disturbance and force. Washing involves rinsing a culture dish with a buffer solution and transferring the solution with animals via glass Pasteur pipette to a new culture plate. This method is rapid and efficient but is not accurate as multiple generations and developmental stages of animals are transferred in bulk. Chemical treatments, such as FUDR, can be dissolved in the culturing media to prevent the production of offspring through blocking DNA replication, and thus, gamete production and egg development. While effective, this method must be applied after developmental maturation as to not disrupt normal developmental processes, this means that there is still a requirement to transfer animals prior to administration. This method also influences multiple cellular signaling pathways, resulting in noticeable effects on the animals as they age (e.g., lifespan extension or altered proteostasis depending on the strain of C. elegans used). Flow cytometry methods automatically sort and transfer individual nematodes from one multi-well plate to another. While this method is very effective and efficient, flow cytometry equipment is prohibitively expensive and inaccessible to many researchers.

Moreover, debris, which may include bacteria from the culture plates of the nematodes, if not removed from a population of nematodes before experimentation, can clog microfluidic devices and can negatively impact behavior assays.

Thus, there is a need within the nematode research and biomedical research community for an affordable, efficient, and accurate method for transferring large numbers of nematodes of the same age (e.g. same size) between culture plates. Moreover, there is a need to remove debris from an age/size synchronized population of nematodes. The invention disclosed herein meets this need providing a device and methods thereof for separating nematodes of the same age (age and size are correlated) from debris and nematodes of other ages and sizes.

### SUMMARY OF THE INVENTION

Herein are provided devices, systems, kits, and methods for separating nematodes by age (i.e., size) from nematodes of a mixed size population. In certain embodiments provided herein, are methods for washing nematodes wherein debris is removed from a desired population of nematodes.

In embodiments are provided a system for separating nematodes by size/age from a mixed size/age population of nematodes and debris, wherein the system comprises an upper housing with a first chamber adapted to receive a liquid sample comprising the mixed size population nematodes, a lower housing with a second chamber, and a filter membrane separating the upper and lower housing. In certain embodiments is provided a system comprising an upper housing with a first chamber adapted to receive a liquid sample comprising the mixed size population of nematodes, a lower housing with a second chamber attached to a first sample vessel and a filter membrane separating the upper and lower housing. In certain embodiments, the system further comprises a second sample vessel attached to the upper housing providing an in-line system for separating a mixed size population of nematodes by size into a first population and a second population of nematodes.

In embodiments, the filter membrane comprises hydrophilic material; a “hydrophilic filter membrane”. In certain embodiments, the filter membrane comprises polyethylene terephthalate (PET), polycarbonate, polystyrene, polypropylene, nylon, Mylar, stainless steel, wire mesh, aluminum, synthetic mesh, plastic, or paper. In other embodiments, the filter membrane comprises a pore size from about 10 μm to about 70 μm. In exemplary embodiments, the filter is located between the upper housing and lower housing and attached directly to the housing. In alternative embodiments, the filter membrane is part of a coupler that is used to attach the filter membrane between the upper and lower housing.

In embodiments, the housing is made of plastic, metal, glass, or non-porous material. In embodiments, the upper housing, lower housing or both are configured for attachment to a sample vessel. In embodiments, the attachment may be via threaded housing and complementary threading on the sample vessel. In other embodiments, the housing may further comprise a flange unit wherein the flange unit is configured for attachment to a sample vessel. In certain embodiments, the sample vessel may comprise a complimentary flange unit for attachment to the flange unit of the upper housing, lower housing, or both.

In embodiments provided herein are methods for using the present system for separating nematodes by size from a mixed size population of nematodes, the method comprising adding a liquid sample comprising the mixed size population of nematodes to a pre-wetted filter membrane within the first chamber of the system; adding a buffer to the filter membrane within the first chamber to draw nematodes that are smaller than a pore size of the filter membrane through the filter membrane; collecting the nematodes that are drawn through the filter membrane in the first sample vessel to provide a first population of nematodes; attaching a second sample vessel to the upper housing and removing the first sample vessel comprising the first population of nematodes from the lower housing; and, collecting the nematodes in the upper housing by adding a buffer to the filter membrane of the second chamber and washing the nematodes from the filter membrane into the second sample vessel, whereby the mixed size population of nematodes is separated by size into a first population and a second population.

### BRIEF DESCRIPTION OF THE DRAWINGS

The drawings illustrate generally, by way of example, but not by way of limitation, various embodiments disclosed herein.

FIG. 1 shows the manufacturing A to F of the device G comprising an upper housing with a first chamber, a lower housing with a second chamber, and a filter membrane separating the upper and lower housing.

FIG. 2 shows use of the device A, wherein a first sample vessel is attached to the lower housing B, adding a liquid sample comprising the mixed size population of nematodes to the first chamber of the upper housing C, attachment of a second sample vessel to the upper housing D, removal of the first sample vessel from the lower housing and inverting the second sample vessel attached to the upper housing E, collecting the nematodes in the upper housing by adding a buffer to the second chamber and washing the nematodes from the filter membrane into the second sample vessel F; collecting nematodes by allowing them to settle to the bottom of the second sample vessel G; and placing collected nematodes on an agar plate H.

FIG. 3 shows a line drawing of the device 100 that includes an upper housing 110 with a first chamber 111 separated from a lower housing 120 with a second chamber 121 by a filter membrane 130. The device 200 is formed by attachment of the lower hosing 120 to a first sample vessel 210, which is removed when the second sample vessel 220 is attached to the upper housing 110.

FIG. 4 shows the motility class distribution of nematodes at days 2, 4, 6, and 8 of adulthood for pick, FUDR, and present device treatment groups. Class A animals moved normally and spontaneously, class B animals moved abnormally and may have required prodding, and class C animals were unable to move. There was no difference between use of the present device, pick and FUDR groups (p>0.05). Three replicates (n=10 animals per treatment plate) were conducted and analyzed with Ordinal logistic model. class B animals. See Example 3. Use of the filter membrane device did not impact motility throughout lifespan

FIG. 5 shows the pharyngeal pump rates of pick, FUDR, and device treatment groups, compared on days 2, 4, 6, and 8 of adulthood. The asterisks denote a significance between the pick, filter membrane method, and FUDR treatment groups for the days specified (*** p<0.05). There was no difference between the filter membrane device and the pick group (p>0.05). Two replicates (N=10 animals per treatment plate) were conducted and analyzed with a one-way ANOVA and a Bonferroni post-test. The bars represent the mean±the standard error of the mean. See Example 3. Use of the filter membrane device did not impact pharyngeal pumping throughout lifespan.

FIG. 6A shows the anterior and FIG. 6B the posterior touch response count of pick, FUDR, and the filter membrane device treatment groups compared on days 2, 4, 6, and 8 of adulthood. Two replicates were conducted with N=10 for each treatment group and compared with a one-way ANOVA and a Bonferroni post-test (p=0.4 and p=0.9 for anterior and posterior, respectively. The bars represent the mean±the standard error of the mean. Use of the filter membrane device did not impact anterior touch response throughout lifespan.

FIG. 7 shows the viable progeny of day-3 adults after a 24 h egg-laying period. The bars represent the mean±the standard error of the mean. N=20-22 animals from at least two separate biological replicates per treatment group. The treatment groups are compared with a t-test, (p>0.05). Use of the filter membrane device did not impact amount of viable progeny on day of adulthood.

FIGS. 8A to D show that use of the filter membrane device did not affect nuclear translocation of DAF-16::GFP, wherein representative images show the DAF-16 translocation of a heat shock group (the positive control) (FIG. 8A), a pick group (the negative control) (FIG. 8B), and a Caenorhabditis Sieve treatment group (FIG. 8C) and a graph representing distribution of the imaged fluorescent signal for each group (FIG. 8D). The positive control group displayed an activation of the DAF-16 nuclear translocation, while use of the filter membrane device did not induce a nuclear translocation and displayed cytosolic fusion protein similar to the nematodes in the negative control group. N=10 animals per treatment group from at least three separate biological replicates. The treatment groups were compared with a one-way ANOVA and a Tukey's post hoc test. The scale bar is 100 μm.

FIG. 9 shows use of the filter membrane device did not affect the expression of hsp16.2::gfp. The HSP-16.2 expressions (in arbitrary fluorescence units) of a heat shock group (the positive control), a pick group (the negative control), and a filter membrane device treatment group are compared. The asterisks denote a high expression of hsp16.2::gfp for the positive control group which was significantly different from the other treatment groups (*** p<0.05). The filter membrane device did not affect the hsp16.2::gfp expression and displayed fluorescence intensities similar to animals in the negative control group (p>0.05). Three replicates were conducted with N=10 for each treatment group and compared with a one-way ANOVA and a Tukey's post hoc test.

FIG. 10 shows use of the filter membrane device did not affect the expression of sod-3::gfp. The SOD-3 expression (in arbitrary fluorescence units) of a 100 μM paraquat group (the positive control), a pick group (the negative control), and the filter membrane device treatment group are shown. The asterisks denote a high expression of sod-3::gfp for the positive control group which was significantly different from the other groups (*** p<0.05). The filter membrane device did not affect the sod-3::gfp expression and displayed fluorescence intensities similar to the animals in the negative control group (p>0.05). Three replicates were conducted with N=10 for each treatment group and compared with a one-way ANOVA and a Tukey's post hoc test.

### DETAILED DESCRIPTION OF THE INVENTION

### Introduction

Provided herein is a system for separating nematodes by size from a mixed size population of nematodes, and methods for using the system for separating the nematodes by size. In certain embodiments, the system and methods may be used to “wash” the nematodes wherein debris are removed from the desired population of nematodes. Size and age are correlated for nematode life cycle, also referred to herein as “life-stage”, thus separating by size also separates the nematodes by age and removes any debris that is smaller than the filter membrane pore size of the system.

Nematodes, especially C. elegans, are a well-studied animal model wherein the complete life cycle includes an egg stage, four larval stages (L1 to L4) and two adult stages (i.e. a young adult stage (mature but not yet reproductively viable) and gravid adult stage (that can bear progeny)), wherein the young adult stage is slightly smaller than the gravid adult stage; and an alternative life stage, the dauer stage. Each of those stages has a diameter in the μm size range, wherein newly hatched larvae are restricted by a pore size of 10 μm and that diameter ranges up to 100 μm during development to gravid adult stage. Uppaluri and Brangwynne A size Threshold Governs Caenorhabditis elegans Developmental Progression, Proc Bio; Sci. 2015 Aug. 22; 282(1813). The size of diameter varies between species, and even between wildtype and mutant or variants of the same species.

The length of the nematodes at different stages is also well known, however the present system and methods separate nematodes by their diameter wherein diameter size and age are correlated. Hence, in embodiments, the filter membrane pore size of the present system separates nematodes by age.

The normal life cycle of a nematode is measured in days from egg to adult and a population quickly becomes mixed as to both size and age. The present system provides a device that efficiently and effectively sorts nematodes of different stages from a large population of mixed size nematodes, while simultaneously removing debris. In certain embodiments, a population of nematodes is synchronized using known chemical treatment methods, and then the present system is used to wash and/or further purify the synchronized population that may become contaminated with different life-stage of nematodes.

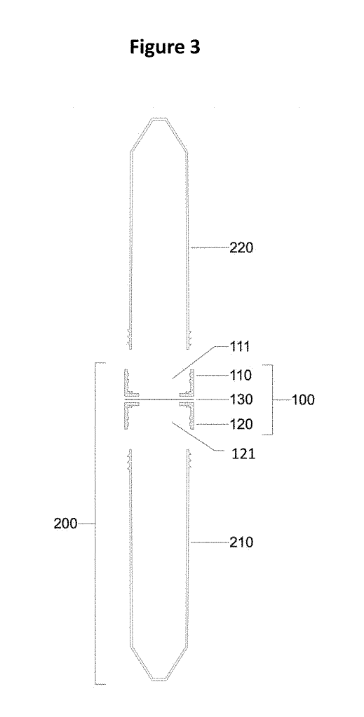

In embodiments are provided a system 100 for separating nematodes by size from a mixed size population of nematodes, wherein the system comprises an upper housing 110 with a first chamber 111 adapted to receive a liquid sample comprising the mixed size population nematodes, a lower housing 120 with a second chamber 121, and a filter membrane 130 separating the upper 110 and lower 120 housing. In certain embodiments, the system further comprises a first sample vessel 210 attached to the lower housing 120 and second chamber 121 forming system 200. See FIG. 3.

In other embodiments provided herein are methods for using the system wherein the steps of the method comprise: a) adding a liquid sample comprising the mixed size population of nematodes to a pre-wetted filter membrane within the first chamber of the system; b) adding a buffer to the filter membrane within the first chamber to draw nematodes that are smaller than a pore size of the filter membrane through the filter membrane; c) collecting the nematodes that are drawn through the filter membrane in the first sample vessel to provide a first population of nematodes; d) attaching a second sample vessel to the upper housing and removing the first sample vessel comprising the first population of nematodes from the lower housing; and, e) collecting the nematodes in the upper housing by adding a buffer to the filter membrane of the second chamber and washing the nematodes from the filter membrane into the second sample vessel, whereby the mixed size population of nematodes is separated by size into a first population and a second population. In embodiments, that method also removes debris from the liquid sample comprising the mixed size population nematodes.

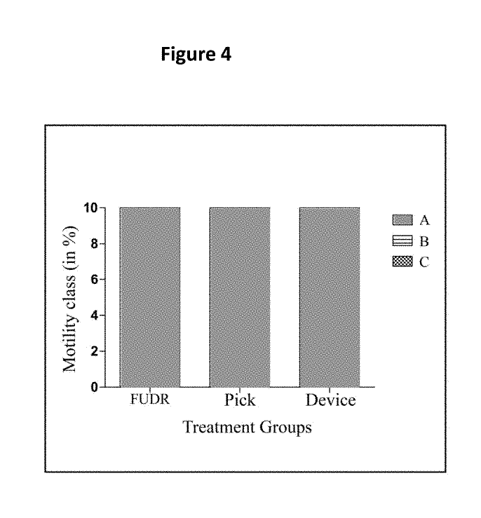

The present system has several advantages to manually picking individual nematodes, washing populations, chemical treatments (e.g. FUDR), and more expensive methods of segregating animals, such as flow cytometry. Use of the present system: 1) has no detectable toxic effects on the healthspan; 2) does not induce genetic stress reporters; 3) does not reduce the number of progeny produced; 4) does not impact motility; 5) does not impact response to mechanical stimulus; and, 6) reduces the amount of a foreign chemical that could influence culture or assays, such as molecular assays, applied to the population of nematodes. See Example 3; FIGS. 4-10; and, Hunter S. et al., Caenorhabditis Sieve: A Low-Tech Instrument and Methodology for Sorting Small Multicellular Organisms. J. of Video Experiments (JoVE); 2018 in-press.

In certain embodiments, use of the system selects nematodes based on body diameter size and can therefore maintain age synchronized groups, wherein eggs, nematodes of a different stage or debris are removed from the desired age synchronized group. Moreover, the invention, as compared to tedious time-consuming methods of picking individual nematodes, is a high throughput method allowing for the rapid transfer and size/age sorting of an entire plate of nematodes. In embodiments, the invention is more effective and efficient than picking individual nematodes.

In addition to those benefits, the present system, device, and methods provide for nematode population maintenance, wherein the system may be used to clean or wash animals (e.g., remove bacterial debris) before use in other experimental applications (e.g., use in microfluidic devices or behavioral assays). For example, with the development of microfluidics to conduct nematode research comes the challenge of keeping microfluidic chambers and microscopic channels free. Often when nematodes are transferred into a microfluidic chip, residue from the bacterial lawn is also transferred. That residue can and often does clog the microfluidic channels making the chip malfunction, which requires cleaning or replacement. Moreover, that residue can also be attractive to the nematodes and can cause aberrations in commonly practiced behavioral experiments, such as chemotaxis assays. The present system, device and methods provide for not only harvesting synchronized animals for microfluidics, but also for cleaning the animals prior to insertion into a microfluidic device and other assays such as behavioral assays. By removing the debris and bacterial residue taken up when collecting animals, the microfluidic channels are less prone to malfunction, thus increasing the operational life of individual chips and the subsequent throughput of research being conducted.

Moreover, while the present device has advantages over known methods for achieving clean, age-synchronized nematodes, other organisms with a micron-sized life-stage may be washed and/or separated using the present system of the disclosure. Other organisms include without limitation, drosophila, or crustaceans (e.g., copepods), including eggs from other organism such as eggs from fish (e.g. zebrafish) or frogs (e.g., Xenopus or Rana). Accordingly, in certain embodiments the filter membrane comprises pores having a size from about 10 μm to about 5000 μm.

### Definitions

As used herein, the terms “a” or “an” are used, as is common in patent documents, to include one or more than one, independent of any other instances or usages of “at least one” or “one or more.”

As used herein, the term “or” is used to refer to a nonexclusive or, such that “A or B” includes “A but not B,” “B but not A,” and “A and B,” unless otherwise indicated.

As used herein, the term “about” is used to refer to an amount that is approximately, nearly, almost, or in the vicinity of being equal to or is equal to a stated amount, e.g., the state amount plus/minus about 5%, about 4%, about 3%, about 2% or about 1%.

As used herein, the term “age” is interchangeable with “size” as the age and size of a nematode are correlated. Unless otherwise stated, “size” as used herein refers to diameter size of the organism. In other words, separating the nematodes by size based on the pore size of the filter screen necessarily separates and provides a population of nematodes that are synchronized by age. As understood herein, the present system separated nematodes based on diameter size, not length of the nematodes, which is also correlated with age.

As used herein, the term “debris” refers to food, bacteria, lint, fibers and/or dust that are removed with the nematodes from their culture plates, and which due to the size are “washed” away from a desired population of nematodes using the present device and methods.

Device

Provided herein are devices and systems (100, 200) for separating nematodes by age and diameter size and simultaneously removing debris, wherein use of the device provides a population of nematodes that are sorted by age and size from a larger mixed size population of nematodes and are clean (e.g., removal of debris). In embodiments the system 100 comprises an upper housing 110 with a first chamber 111 adapted to receive a liquid sample comprising the mixed size population nematodes, a lower housing 120 with a second chamber 121, and a filter membrane 130 separating the upper and lower housing. In certain embodiments, the system comprises an upper housing 110 with a first chamber 111 adapted to receive a liquid sample comprising the mixed size population of nematodes, a lower housing 120 with a second chamber 121 attached to a first sample vessel 210 and a filter membrane 130 separating the upper and lower housing. In certain embodiments, the system further comprises a second sample vessel 220 attached to the upper housing providing an in-line system for separating a mixed size population of nematodes by size into a first population and a second population of nematodes.

In embodiments, the device comprises a vessel with two openings (e.g., an upper housing and a lower housing) and a filter membrane spanning part or all of the cross-sectional area of the housing such that any object entering one opening (e.g., upper housing with a first chamber adapted to receive a liquid sample comprising the mixed size population nematodes) could not exit through the other opening (e.g., lower housing with a second chamber) without passing through the filter membrane. In embodiments, the housing, upper and/or lower housing (110, 120), is made of, and/or comprises, plastic, metal, glass, or non-porous material. No intended limitation is provided herein for the material used to manufacture the housing, wherein the housing comprises a material that provides a rigid structure and is adapted or configured to receive a liquid sample comprising a mixed size population of nematodes. In certain embodiments, the upper and/or lower housing, or any portion of a wall of the housing that the liquid sample may contact, may be configured as straight, flat, and/or smooth.

In embodiments, the device is configured to be used with one or more sample-retaining vessels (210, 220), which can be any size or shape vessel capable of retaining a liquid sample. In embodiments, the upper housing, lower housing, or both are configured for attachment to a sample vessel. In certain embodiments, the sample-retaining vessels are conical plastic tubes with threads for attachment to the upper and/or lower housing. In embodiments, the attachment to a sample vessel is via threaded housing and complementary threading on the sample vessel. In other words, the upper housing, lower housing, or both, comprise threads that are complimentary to the threads of the sample vessel wherein the sample vessel may be attached to the housing via counter clockwise threading of the sample vessel to the upper or lower housing.

In certain other embodiments, the upper housing, lower housing, or both comprise a flange unit configured for the attachment to a sample vessel. A flange unit, as understood by one of skill in the art, is an external or internal ridge or rim (e.g. a lip) that can be used to attach a component with a similar diameter, such as a sample vessel, to the upper or lower housing. In this instance, the flange unit on the upper or lower housing would be attached to a flange unit on a sample vessel using pins, screws, bolts, threads or another comparable mechanism to attach two flange units together. In embodiments, a sample vessel is attached to the upper or lower housing via a flange unit, or via complementary threads or other mechanisms for attaching components of a similar diameter together.

In embodiments, the filter membrane comprises polyethylene terephthalate (PET), polytetrafluoroethylene (PTFE), polycarbonate, polystyrene, polypropylene, nylon, Mylar, stainless steel, wire mesh, aluminum, synthetic mesh, plastic, or paper. In certain embodiment, the filter membrane comprises a hydrophilic material. In exemplary embodiments, the filter membrane is a nylon membrane. In embodiments, the filter membrane comprises a pore size 2 to 5 μm smaller than the diameter size of the desired age of the nematode to be separated from a mixed size population. In certain embodiments, such as N2 isolate of C. elegans, eggs have a size of about 30 μm, L1 a size of about 11.7±0.2 μm (n=55)), L2 a size of about 17.0±0.2 μm (n=49), L3 a size range from 22.2±0.3 μm (n=92), L4 a size of about 29.6±0.6 μm (n=16), young adult stage a size of about 47.9±0.8 μm (n=33) and a reproductive adult stage with a maximum diameter of about 80 μm. Maguire S M. et al., The C. elegans Touch Response Facilitates Escape from Predacious Fungi, 9 Aug. 2011, Current Biology; 21(15): 1326-1330; So S. et al., Control of body Size in C. elegans Dependent on Food and Insulin/IGF-1 Signal, Genes to Cells (2011) 16, 639-651; Hirose T. et al., Cyclic GMP-dependent Protein Kinase EGL-4 Controls Body Size and Lifespan in C. elegans, 2003 Devel. 130:1089-1099; C. elegans II. 2nd edition, Riddle D L, Blumenthal T, Meyer B J, et al., editors. Cold Spring Harbor (N.Y.): Cold Spring Harbor Laboratory Press; 1997; and, Brown, J. D. (2009) Synchronization and Media Exchange in Large-Scale Caenorhabditis elegans Cultures. All Graduate Theses and Dissertations, 974.

There is variability in the diameter size ranges for the different life-stages of nematodes, between species, and within a species including variants and mutants of wild-type, such as C. elegans. The above ranges are only one representative size range for each stage (N2 isolate of C. elegans), wherein there could be variability of ±2 μm for each stage. The published diameter measurements for nematode eggs, however have a large degree of variability and range from 24 μm to 45 μm. L'Hernault, S. W., Shakes, D. C., and Ward, S. (1988). Developmental Genetics of Chromosome I Spermatogenesis-Defective Mutants in the Nematode Caenorhabditis elegans. Genetics 120, 435-452; Sofela, S. et al. (2018) High-Throughput Sorting of Eggs from Synchronization of C. elegans in a Microfluidic Spiral Chip. Lab Chip, DOI: 10.1039/C7LC00998D; Harvey S. C. And Orbidans H. E. (2011) All Eggs are Not Equal: The Maternal Environment Affects Progeny Reproduction and Developmental Fate in Caenorhabditis elegans. PLoS One, 6(10).

In embodiments, the filter membrane comprises a pore size that is about 2 to 5 μm smaller than the desired nematode population, wherein the debris and smaller non-desired nematodes passes through the filter membrane and the desired nematode population is retained on the filter membrane surface of the upper housing. The slightly smaller pore size, as compared to the desired nematode diameter size, ensures nematodes will not inadvertently squeeze through the filter membrane lowering the recovery of the desired nematode population, including with any slight pressure that may be applied during the present methods. In embodiments, the diameter size of the desired life-stage nematodes is empirically determined and a filter membrane with a particular pore size selected based on the average size of the measured diameter sizes. In certain other embodiments, the average diameter size of a desired life-stage is derived from publications and used to select the appropriate filter membrane with a particular pore size. It is understood one of skill in the art knows how to determine an average diameter size for a desired nematode population and select an appropriate filter membrane with a particular pore size.

In embodiments, the filter membrane comprises a pore size from about 10 μm to about 1000 μm. In certain embodiments, the filter membrane comprises a pore size from about 20 to μm wherein use of will separate eggs, L1 to L4 and debris from the adult stages. In embodiments, the filter membrane comprises a pore size from about 10 μm to about 75 μm, from about 15 μm to about 30 μm, or from about 30 μm to about 65 am. In embodiments, the filter membrane comprises a pore size about 10 μm, about 12.5 μm, about 15 μm, about 17.5 μm, about 20 μm, about 22.5 μm, about 25 μm, about 27.5 μm, about 30 μm, about 32.5 μm, about 35 μm, about 37.5 μm, about 40 μm, about 42.5 μm, about 45 μm, about 47.5 μm, about 50 μm, about 52.5 μm, about 55 μm, about 57.5 μm, about 60 μm, about 62.5 μm, about 65 μm, about 67.5 μm, or about 70 am.

In certain other embodiments, the filter membrane comprises a pore size to isolate nematodes in the L1 stage, L2 stage, L3 stage, L4 stage, immature adult stage or adult stage. In embodiments, the filter membrane comprises a pore size to separate the adult stages from eggs and L1 to L4 stages of the mixed size population of nematodes. In embodiments, the filter membrane comprises a pore size to separate debris from any stage of the nematode life cycle.

In exemplary embodiments, the filter membrane comprises a flat nylon membrane. Pore size of the membrane may be selected to separate animals of different life stages. In certain embodiments, a 20 μm pore size is appropriate for separating developing embryos and larval stages smaller than the fourth larval stage (L4; average body diameter of 29 to 32 μm) from adult stages. A 50 μm pore size will allow all other life stages aside from adults (average body diameter of 49 to 80 μm) to be isolated.

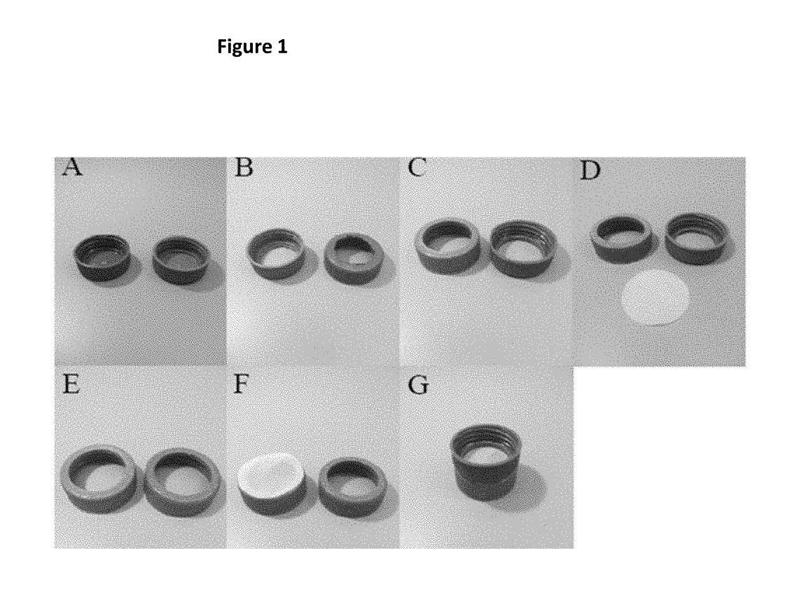

In embodiments, the filter membrane 130 is permanently and directly secured between the upper 110 and lower 120 housing providing a system with an individual filter membrane 130 comprising a specific pore size. See FIGS. 1 and 3. In certain other embodiments, the system is modular as to the filter membrane and corresponding pore size, wherein the filter membrane is first attached to a coupler that may be attached to the upper and lower housing to form the present system. In this instance, the coupler may comprise threads and/or a flange unit, that are used to attach the coupler and filter membrane to the upper and lower housing. The filter membranes may then be interchangeable allowing a user to use one filter membrane with a specific pore size, and then use a different pore size filter to either further separate the sample or to isolate nematodes from a different sample based on a different size.

In embodiments, the liquid sample comprises a mixed size population of nematodes and an aqueous solution. In embodiments, the aqueous solution comprises a buffer, such as M9. See Example 2. In certain embodiments, the aqueous solution comprises a saline solution. In certain embodiments, the liquid sample may further comprise debris (e.g., food and/or bacteria) from the nematode growing media. In certain embodiments, the mixed size population of nematodes may be previously age synchronized using well-known chemical treatment protocols, such as dissolving gravid hermaphrodites in a bleaching solution providing a population of eggs that develop to a desired stage. In that instance, the age synchronized nematode population may contain a majority of one life-stage and debris; use of the present system would further separate those contaminating life-stage nematodes and wash away any debris.

The device or system may be any shape that allows the present methods to be performed successfully. In embodiments, the shape will have simple geometry that allows nematodes to pass through with little to no obstruction other than from the filter membrane. In embodiments the present system will have the upper and lower housing located opposite each other, or in-line. See FIGS. 1, 2 and 3. In certain embodiments, the upper housing, lower housing and filter screen are located in parallel planes providing an in-line system. See FIG. 3.

In embodiments, the device may be any overall size that allows the method to be performed successfully. In embodiments, the size of the device and/or system allows for easy manual handling and use on a bench in a laboratory. In certain embodiments, the size of the upper and/or lower housing will minimize the distance between each open end of the housing without compromising the ability of the device to be attached to a sample vessel.

Methods

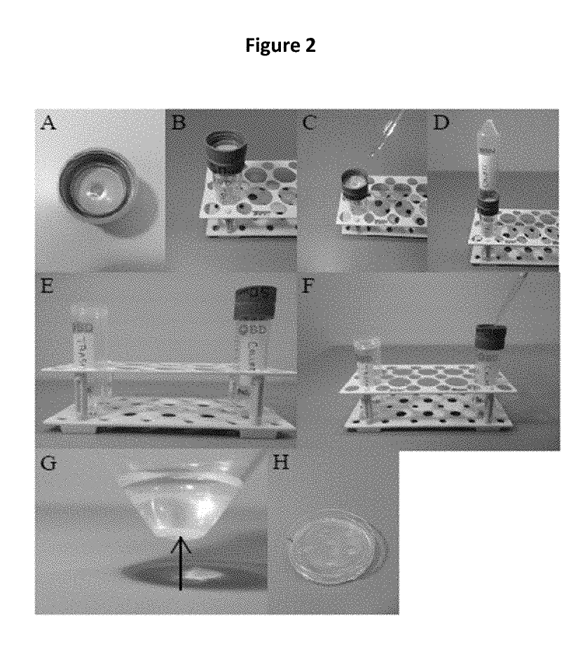

Provided herein are methods for separating nematodes of a desired size and/or age from a mixed size population of nematodes and/or debris using the system and device of the present disclosure. In certain embodiments, methods for separating nematodes by size (and age) from a mixed size population of nematodes comprises the steps of a) adding a liquid sample comprising the mixed size population of nematodes to the filter membrane within the first chamber of the present system; b) adding a buffer to a pre-wetted filter membrane within the first chamber to draw nematodes and/or debris that are smaller than a pore size of the filter membrane through the filter membrane; c) collecting the nematodes and/or debris drawn though the filter membrane in the first sample vessel to provide a first population of nematodes; d) attaching a second sample vessel to the upper housing and removing the first sample vessel comprising the first population of nematodes from the lower housing; and, e) collecting the nematodes (desired population size) in the upper housing by adding a buffer to the filter membrane of the second chamber and washing the nematodes from the filter membrane into the second sample vessel, whereby the mixed size population of nematodes is separated by size into a first population and a second population. See Example 2 and FIG. 2. In certain embodiments, the method also removes debris from the liquid sample comprising the mixed size population of nematodes, wherein the second population of nematodes is free of debris.

In certain embodiments, the above steps are repeated with a second filter membrane comprising a different pore size, for example when separating L4 stage nematodes from a mixed size population that contains L1 to L4 and adult stage nematodes. In that instance, the steps of the method would be performed with a membrane filter comprising a pore size about 2 to 5 microns smaller than the average diameter of the L4 nematodes, wherein the size may be empirically determined or based on published diameter sizes. The L1 to L3 stage nematodes would be drawn through the filter membrane into the first sample vessel, and optionally discarded, while the population comprising L4 and adult stages would be washed into the second sample vessel for further separation. The steps of the method would then be repeated with the L4 and adult mixed size population with a filter membrane comprising a pore size between the L4 and adult stages, or 2 to 5 microns smaller than the young adult stage, wherein the desired L4 population would be drawn through the filter membrane to the first sample vessel and the adult stages would be washed into the second sample vessel. In embodiments, similar methodology may be used to isolate, separate and/or wash nematodes with an intermediate size in a population comprising un-desired nematodes and/or debris that is both larger and smaller than the desired diameter sized nematode population.

In embodiments, the filter membrane is pre-wetted with buffer or aqueous solution before adding the liquid sample of step a). As one of skill in the art understands, that is a standard procedure, to avoid any inadvertent binding of material to be separated to the filter membrane. Moreover, without proper pre-wetting, fluids may pool on the top surface of the filter membrane, wherein surface tensions are generally great enough to prevent fluid from entering the pores. This may be particularly problematic with small pore sizes and materials that are relatively hydrophobic. In embodiments, pre-wetting is required so that the spaces within the pores become fluid filled, and a droplet forms below the filter membrane.

In embodiments, the second population (e.g. the desired sized nematodes) are collected in the second sample vessel and allowed to settle to the bottom of the sample vessel. See FIG. 2G. To aggregate the nematodes, the sample vessel may be subjected to a gentle spin. In alternative embodiments, the sample vessel is placed in a holder and the nematodes allowed to settle to the bottom over time without the use of a centrifuge or rotating force.

In certain embodiments, the nematodes of step b) are drawn through the pre-wetted filter membrane via gravity with no additional force applied. The pre-wetting of the filter membrane provides a drop of fluid (e.g. aqueous buffer) that coalesces below the filter membrane via gravity, which draws the fluid of the sample (e.g., liquid sample comprising the mixed size population nematodes) down through the filter membrane. In other words, fluid is pulled through the filter membrane by the forces of surface tension acting within the fluid.

In certain other embodiments, the drawing of the nematodes through the filter membrane is aided with subjecting the sample vessel to a rotating force. One of skill in the art understands this would be a slow or gentle rotating force to avoid forcing nematodes through the filter membrane that are intended to be retained above the filter membrane. In other embodiments, the drawing of the nematodes through the filter membrane is aided by wicking fluid away from the lower surface of the filter membrane, such as with use of an absorbent material. That process draws more of the liquid sample comprising the mixed size population nematodes though filter membrane, but only those nematodes with a diameter smaller than the pore size of the filter membrane will be drawn through the filter membrane.

### EXAMPLES

The following examples are put forth to provide those of ordinary skill in the art with a complete disclosure and description of how to use the embodiments provided herein and are not intended to limit the scope of the disclosure nor are they intended to represent that the Examples below are all of the experiments or the only experiments performed. Efforts have been made to ensure accuracy with respect to numbers used (e.g. amounts, temperature, etc.) but some experimental errors and deviations should be accounted for. It should be understood that variations in the methods as described can be made without changing the fundamental aspects that the Examples are meant to illustrate.

### Example 1: Design and Fabrication of the Device

Provided herein is system 100 for separating nematodes by size from a mixed size population of nematodes, comprising an upper housing 110 with a first chamber 111 adapted to receive a liquid sample comprising the mixed size population nematodes, a lower housing 120 with a second chamber 121, and a hydrophilic filter membrane 130 separating the upper housing 110 and lower housing 120. See FIGS. 1 and 3

An exemplary system was designed and prepared using 50 ml conical tubes (sample vessel), their lids, and a nylon membrane as the filter membrane 130. Two lids were acquired from 50 mL conical tubes and the center area inside the inner lip of the lids (when viewed from the bottom) was removed using a Bunsen burner and a hot metal probe or a soldering iron or stepped drill bit. See FIGS. 1B and 1C. The cut edges and top surface were cleaned and sanded with a curved file or a rotary grinding tool (e.g., Dremel). A circle of monofilament mesh 130 was cut to the appropriate diameter. For this, a lid was traced onto a monofilament nylon mesh sheet and cut just inside the line drawn. See FIG. 1D.

Grooves/slashes were cut into the top surface of the lids to enhance the adhesion of the two lids when glue was applied to the plastic. The lids were then cleaned with ethanol and allowed to dry. Cyanoacrylate glue was applied to the top surface of both lids, keeping to the outer edge. The monofilament mesh was placed onto one lid (e.g. lower housing 120, which include the second chamber 121). The second lid (e.g. upper housing 110, which includes the first chamber 111) was placed inverted on top of the filter membrane 130. The upper 110 and lower housing 120 were pressed firmly together while ensuring that the filter membrane 130 was taut. See FIGS. 1F and 1G. Once the initial layer of glue was dry, a ring of cyanoacrylate glue was applied around the outer gap between the lids.

A 50 ml conical tube (e.g. sample vessel 210) was attached to the lower housing 120 to form an exemplary system 200. See FIG. 2B. Provided herein is an exemplary system 200 for separating nematodes by size from a mixed size population of nematodes, comprising an upper housing 110 with a first chamber 111 adapted to receive a liquid sample comprising the mixed size population of nematodes, a lower housing 120 with a second chamber 121 attached to a first sample vessel 210 and a filter membrane 130 separating the upper 110 and lower housing 120.

### Example 2: Methods of Separating Nematodes by Size from a Mixed Size Population of Nematodes

Provided herein is a method for separating nematodes by size using the device designed and fabricated in Example 1. See FIG. 2.

The device was attached to a 50 ml conical tube and the filter membrane, nylon screen (20 μm and 50 μm) was pre-wet by pipetting a saline solution of M9 (5 g NaCl+6 g Na2HPO₄+3 g KH₂PO₄+1 L ultrapure diH₂O+1 ml MgSO₄ (1M) onto center of the filter membrane until a droplet formed, condensed, and drips off bottom of filter membrane. Brenner, S. The genetics of Caenorhabditis elegans. Genetics. 77 (1), 71-94 (1974).

Following the pre-wetting step, a mixed size population of nematodes was added to the filter membrane via the first chamber of the upper housing. See FIG. 2C. The mixed size population of nematodes was obtained by washing a population of nematodes from a culture plate by washing the culture plate with M9 buffer (we used 3 ml of M9 buffer for a 60 mm culture plate to sufficiently wash most of the nematodes from the culture plate). Nematodes were cultured on a standard Nematode Growth Media (NGM) 1 L of NGM consists of 2.5 g of peptone, 17 g of agar, 3 g of NaCl, 975 mL of double-distilled water, 1 mL of 5 mg/mL cholesterol, 1 mL of 1 M CaCl₂, 1 mL of 1 M MgSO₄, 25 mL of 1 M KHPO₄, and 0.5 mL of 100 mg/mL streptomycin) at 25° C. Brenner, S 1974.

About 1 ml of the wash comprising the mixed size population of nematodes was added to the center of the pre-wet filter membrane via the first chamber of the upper housing using a glass pipette to minimize the number of worms lost during the pipetting process. Subsequent 1 ml aliquots of the mixed size population of nematodes was added to the filter membrane until all the wash from the culture plate was added to the device.

Following addition of the entire mixed size population of nematodes from one culture plate to the filter membrane via the first chamber of the upper housing of the device, a rinse of M9 buffer was added to the filter membrane to aid in flushing small larvae, eggs, food, bacteria, and other debris through the filter membrane to the first sample vessel (50 ml conical tube) attached to the lower housing. The rinse was repeated to ensure only those nematodes larger than the pore size of the filter membrane remained on the filter membrane in the first chamber of the upper housing.

Next, size-matched nematodes were harvested from the filter membrane, wherein the first sample vessel attached to the lower housing and comprising the debris that washed through the filter membrane was removed, a second sample vessel was attached to the upper housing and inverted (flipped quickly to keep droplet comprising the size-matched nematodes from migrating) so that the second sample vessel is below the device. Alternatively, an absorbent material may be used to wick fluid from the bottom of the filter screen prior to flipping. That eliminates the need for the flipping to invert second sample vessel and upper housing to be done quickly. The filter membrane of the lower housing (now topside) was rinsed, operating in the center of the filter membrane, and maintaining the droplet) with M9 buffer to wash the size-matched nematodes in to the second sample vessel. The nematodes were allowed to settle to the bottom of the sample vessel (they can also be gently spun down (e.g., <1000 rpm) for approximately 1 min.) to form a pellet. The M9 buffer was aspirated down to about less than 0.5 ml to remove as much fluid as possible without disturbing the nematode pellet. The nematodes were pipetted with a glass Pasteur pipette onto a culture plate and allowed to dry. See FIG. 2H wherein multiple droplets were spaced on the plate to speed up the drying process.

To quantify how effective the sieve is at sorting C. elegans, age-synchronous N2 nematodes were grown to day 1 of adulthood at 25° C. (i.e., 48 h after egg laying) and then transferred to fresh NGM plates (totaling N=50 or N=100 animals per treatment group).

After 24 h of recovery, the nematodes were transferred to new NGM plates following separation using the present device, wherein the nematodes were counted to calculate the number successfully separated and transferred. To establish the percentage yield of the present device, two devices were tested, as disclosed above, with 20 μm or 50 μm pore-size filter membranes. A mean yield of a >90% successful animal transfer was achieved for both pore sizes tested.

TABLE 1
Recovery Results of Nematodes after Use of the Device

| Pore size | N = 50 | N = 100 |
|---|---|---|
| 20 μm | 95.33% | |
| 50 μm | 99.00% | 93.33% |

### Example 3: Comparison of Three Methods for Separating Nematode by Size: Individual Picking, Chemical Treatment and Size Exclusion Using the Device of Example 1

The three groups tested were separated by: 1) individual picking, wherein nematodes were selected and transferred manually using a platinum loop; 2) the chemical treatment fluorodeoxyuridine (FUDR), wherein 100 mg/mL FUDR was added to the NGM media to a final concentration of 100 μM to prevent any progeny production, and then the nematodes were transferred every other day to a fresh NGM plate to avoid food depletion; and, 3) the device of Example 1. The three test groups were subject to phenotypic assays demonstrating use of the present device does not impact the health span metrics disclosed below.

Motility:

In C. elegans the normal sinusoidal movement (i.e., motility) declines with age and is a marker of overall health. To determine if the present device influenced motility, motility scores were compared for pick, FUDR, and present filter membrane device treatment groups on days 2, 4, 6, and 8 of adulthood. The mobility assay was performed as disclosed by Hendon et al. Stochastic and genetic factors influence tissue-specific decline in ageing C. elegans. Nature. 419 (6909), 808-814 (2002). The nematodes were assigned a motility score and assigned to a class (A, B, or C). Class A individuals move spontaneously in a normal, sinusoidal pattern. Class B individuals move in markedly non-sinusoidal movements and may require prodding to encourage movement. Class C individuals move their head and/or tail in response to prodding but are unable to move across the agar.

All the nematodes across every group (n=10/group) exhibited normal and spontaneous movement patterns (Class A) at multiple ages throughout adulthood (days 2, 4, 6, and 8 of adulthood; p>0.05 for multiple comparisons on all days, FIG. 4).

Pharyngeal Pump Rate:

The ability of C. elegans' pharyngeal muscles to pump declines with age and is another biomarker of healthspan. To determine if the present filter membrane device influenced the nematodes' pharyngeal pump rate, the three treatment groups were compared on days 2, 4, 6, and 8 of adulthood (n=8 to 10 per group). The grinder movement of the nematode's terminal pharyngeal bulb was visually counted under a stereomicroscope at a 600× final magnification for 1 minute, wherein a statistical analysis with a one-way ANOVA with α=0.05 and Bonferroni post-tests with α=0.05 was performed.

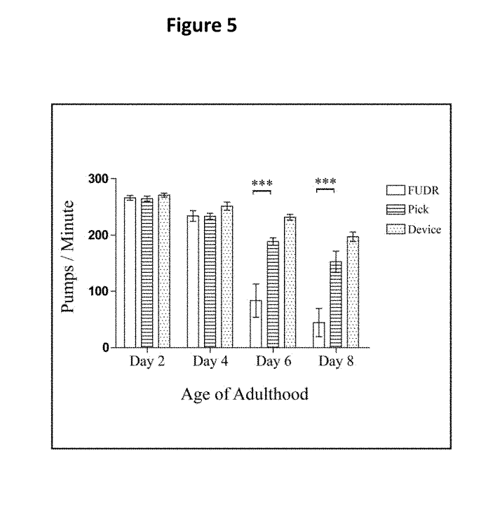

There was a significant difference between the animals that underwent the picking and FUDR methods on days 6 (p<0.001) and 8 (p<0.001). Also, there was a significant difference between the present device and FUDR groups on days 6 (p<0.001) and day 8 of adulthood (p<0.001). However, there was no statistically significant difference between the pick and the present device groups for any day (p>0.05, FIG. 5), indicating that the sieve does not impact this measure of healthspan.

Gentle Touch Response:

Response to mechanical stimulus is a physiological marker to assess aging or general health; thus, the impact of different transfer methods on both the anterior and the posterior gentle touch responses were tested. Responses to a gentle touch were compared between the three treatment groups, wherein the assay was performed based on the methods described by Calixto et al. Enhanced neuronal RNAi in C. elegans using SID-1. Nature Methods. 7 (7), 554-559 (2010). The anterior and posterior touch response was recorded by gently stroking an eyelash pick perpendicularly across the tail or the head (5× each, alternating head and tail) of the animals. Any movement in the opposite direction of the stroke was scored as 1 point on a scale of 0 to 5 for both the anterior and the posterior response. A statistical analysis with a one-way ANOVA with α=0.05 and Bonferroni post-tests with α=0.05 was conducted using the recorded scored.

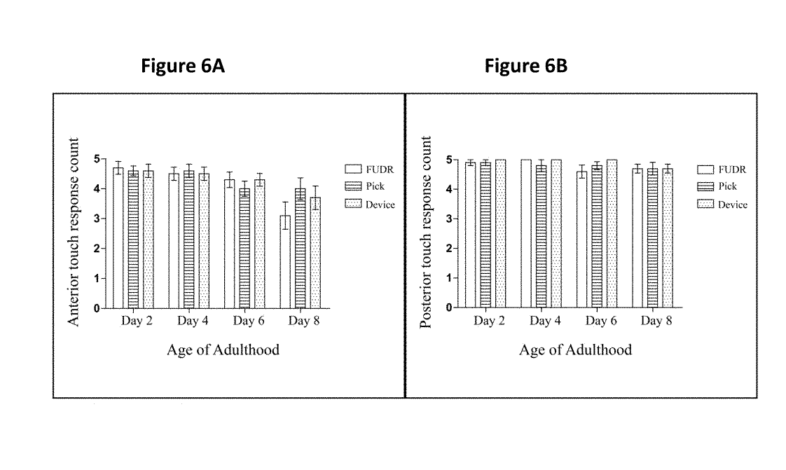

There was no statistically significant difference between the pick, FUDR, and present filter membrane device treatment groups (n=8/group), either anteriorly or posteriorly, for any day of testing (p>0.4 for all comparisons; FIGS. 6A and 6B).

Fecundity: To establish if the present device influenced the amount of viable progeny produced by C. elegans (reproductive health), the individual offspring produced in a 24 h period during day 3 of adulthood were counted and compared (n=20 to 22 per group). Age-synchronous N2 nematodes were grown to day 2 of adulthood at 25° C., and then 60 h after egg lay, nematodes were transferred to new NGM plates with either a platinum pick or the present device and given 4 h to recover, wherein the nematodes in the present device treatment group were dried for 20-30 min. After recovery, the nematodes were individually plated via an eyelash pick to NGM plates, give 24 h to lay eggs, and then nematodes removed. The eggs (progeny) developed under normal conditions at 25° C. for another 24 h.

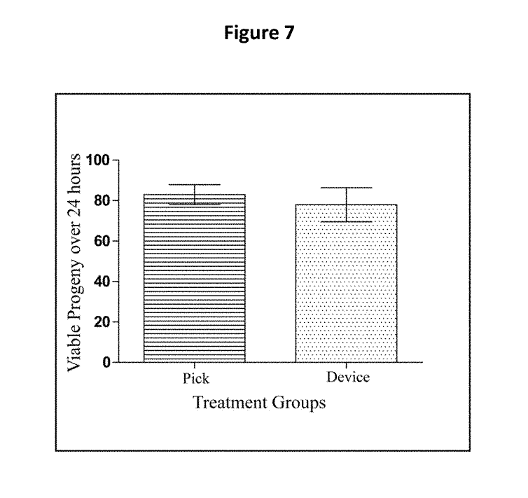

The number of viable F1 generation individuals was counted and statistical analysis using a T-test with α=0.05 was performed. Viable offspring were eggs that successfully hatched and began their development through the regular larval cycles. The use of the Caenorhabditis Sieve did not significantly impact the number of progeny produced when compared to a pick treatment group (p=0.61, FIG. 7).

Fluorescent Reporter Stress Response Assays:

Three commonly used fluorescent reporter assays were performed to detect potential markers of stress: a DAF-16::GFP translocation into the nuclei of cells in a strain [TJ356-zIs356 (pDAF-16::DAF-16-GFP;rol-6)]; an hsp-16.2 expression [TJ375-gpls 1 (hsp-16.2p::GFP)]; and a sod-3 expression [CF1553-muIs84([pAD76]sod-3p::GFP+rol-6[su1006])]. Henderson, S. T., Johnson, T. E. daf-16 integrates developmental and environmental inputs to mediate aging in the nematode Caenorhabditis elegans. Current Biology. 11 (24), 1975-1980 (2001); Rea, S. L., Wu, D., Cypser, J. R., Vaupel, J. W., Johnson, T. E. A stress-sensitive reporter predicts longevity in isogenic populations of Caenorhabditis elegans. Nature Genetics. 37 (8), 894-898 (2005); and, Libina, N., Berman, J. R., Kenyon, C. Tissue-specific activities of C. elegans DAF-16 in the regulation of lifespan. Cell. 115 (4), 489-502 (2003). All strains of C. elegans were obtained from the Caenorhabditis Genetics Center (CGC), which is supported by the National Institutes of Health—Office of Research Infrastructure Programs (P40 OD010440).

For each assay, age-synchronous nematodes from the three treatment groups were cultured at 20° C. and examined at day 3 of adulthood. For the experiments: the negative control was transferred daily manually with a platinum pick; the positive control was transferred daily manually with a platinum pick plus an established stressor; and, the nematodes washed with the present filter membrane device were passed through the filter membrane and allowed to recover for 30 min on NGM just before imaging. The positive control animals were heat shocked at 37° C. for 30 min before imaging (DAF-16::GFP assay) and for 90 min 20 h before imaging (hsp-16.2 assay). The positive controls, for the sod-3 assay, were treated with 100 mM paraquat for 4 h before imaging.

At day 3 of adulthood, the nematodes were harvested with an eyelash pick and mounted to a coverslip with 1 μL of a 36% poloxamer 407 surfactant solution to immobilize the nematodes. The mounted nematodes were sandwiched with another coverslip and then mounted on a standard microscope slide, wherein the nematodes were imaged using an 8× magnification on an inverted fluorescent microscope (for an overall magnification of 80×) using a constant exposure with a FITC filter. Differences in the fluorescent localization between the three groups was recorded and results compared using an ordinal logistics statistical model in statistical analysis software (DAF-16::GFP assay) and for the hsp-16.2 and the sod-3 expression assays, a one-way ANOVA with α=0.05 and Tukey's post hoc tests with α=0.05 was used to compare the total fluorescence of the head region.

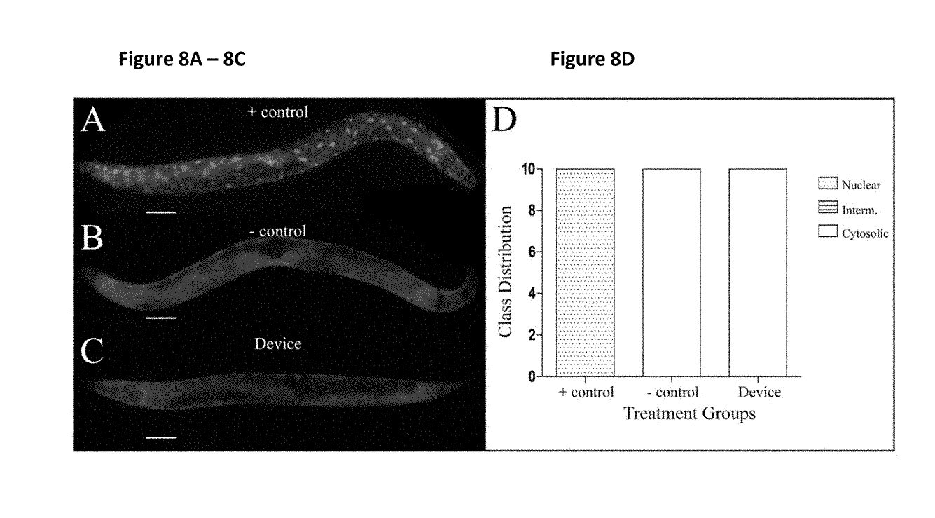

In C. elegans, the activation of the transcription factor DAF-16 is associated with increased stress resistance. The nuclear localization of DAF-16 was examined in a transgenic nematode strain TJ356, which expresses DAF-16 fused to a green fluorescent protein (DAF-16::GFP). Under normal growth conditions, DAF-16::GFP is localized primarily in the cytosol, but under various stressors (e.g., heat stress), it is rapidly translocated into the nucleus. To test the impact of sorting with the present device on DAF-16 translocation, DAF-16::GFP localizations were compared in age-matched day-5 adults in a positive control group (heat stress), a negative control group (manual transfer via pick), and a present device treatment groups (n=10/group). The transfer (e.g. transfer through filter membrane and then plating) with the present device did not affect the nuclear translocation of DAF-16::GFP, and showed a similar phenotype to the negative control animals (p>0.05, FIG. 8).

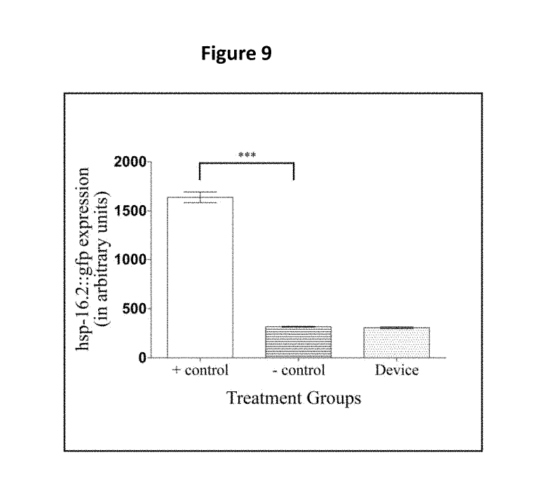

Small heat shock proteins like HSP-16.2 are biomarkers of a stress response, and they are highly expressed during an exposure to heat shock or oxidative stress agents. The TJ375 strain has a GFP reporter gene fused with an hsp-16.2 promoter gene that is not active under normal conditions. However, after an exposure to a heat shock, HSP-16.2 protein expression is induced, and the animals display high levels of GFP expression. To test the involvement of the present device on an HSP-16.2-mediated stress response, the fluorescence density in the pharynx region (n=10 animals/group) of age-matched animals was compared in day-5 adults between a positive control group (heat stress), a negative control group (picking), and a present device treatment group. The transfer with the present device did not significantly induce the expression of HSP-16.2::GFP (hsp-16.2::gfp) when compared to the negative control (p>0.05, FIG. 9).

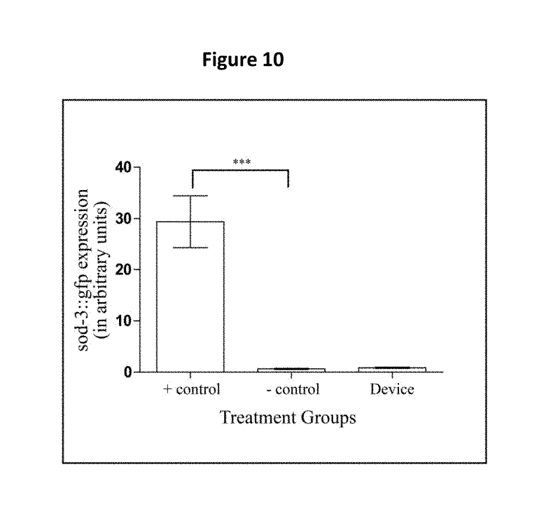

In C. elegans, the anti-oxidant gene that codes for superoxide dismutase 3 (SOD-3) is up-regulated during oxidative stress. The C. elegans strain CF1553 expresses green fluorescent protein (GFP)-labeled SOD-3, whose expression is induced by oxidative stressors, such as paraquat. To test the involvement of the present device sorting on the antioxidant response in C. elegans, the fluorescence density in the head region of age-matched day-5 adults was compared between a positive control population (100 μM paraquat treatment), a negative control population (manual picking), and a present device-transferred population. The transfer with the present device did not significantly induce the expression of sod-3::gfp when compared to the negative control (p>0.05, FIG. 10).

All references cited herein are herein incorporated by reference in entirety.

We claim:

1. A system for separating nematodes by size from a mixed size population of nematodes, comprising:
an upper housing with a first chamber adapted to receive a liquid sample comprising the mixed size population nematodes, a lower housing with a second chamber, and a hydrophilic filter membrane separating the upper and lower housing.

2. The system of claim 1, wherein the housing is made of plastic, metal, glass, or non-porous material.

3. The system of claim 1, wherein the lower housing, upper housing, or both comprise a flange unit.

4. The system of claim 3, wherein the flange unit is configured for attachment to a sample vessel.

5. The system of claim 1, further comprising a coupler disposed between the upper and lower housing and comprising the membrane.

6. The system of claim 1, wherein the upper housing, lower housing, or both are configured for attachment to a sample vessel.

7. The system of claim 6, wherein the attachment to a sample vessel is via threaded housing and complementary threading on the sample vessel.

8. The system of claim 6, further comprising a first sample vessel attached to the lower housing.

9. The system of claim 6, further comprising a second sample vessel attached to the upper housing.

10. The system of claim 1, wherein the filter membrane comprises a pore size from about 15 μm to about 65 μm.

11. The system of claim 1, wherein the filter membrane comprises a pore size from about 15 μm to about 30 μm.

12. The system of claim 1, wherein the filter membrane comprises a pore size from about 30 μm to about 65 μm.

13. The system of claim 1, wherein the filter membrane comprises polyethylene terephthalate (PET), polycarbonate, polystyrene, polypropylene, nylon, Mylar, stainless steel, wire mesh, aluminum, synthetic mesh, plastic, or paper.

14. A system for separating nematodes by size from a mixed size population of nematodes, comprising:
an upper housing with a first chamber adapted to receive a liquid sample comprising the mixed size population of nematodes, a lower housing with a second chamber attached to a first sample vessel and a filter membrane separating the upper and lower housing.

15. The system of claim 14, further comprising a second sample vessel attached to the upper housing providing an in-line system for separating a mixed size population of nematodes by size into a first population and a second population of nematodes.

16. A method for separating nematodes by size from a mixed size population of nematodes, comprising:
a. adding a liquid sample comprising the mixed size population of nematodes to a pre-wetted filter membrane within the first chamber of the system according to claim 14;
b. adding a buffer to the filter membrane within the first chamber to draw nematodes that are smaller than a pore size of the filter membrane through the filter membrane;
c. collecting the nematodes drawn through the filter membrane in a first sample vessel to provide a first population of nematodes;
d. attaching a second sample vessel to the upper housing and removing the first sample vessel comprising the first population of nematodes from the lower housing; and,
e. collecting the nematodes in the upper housing by adding a buffer to the filter membrane of the second chamber and washing the nematodes from the filter membrane into the second sample vessel,
whereby the mixed size population of nematodes is separated by size into a first population and a second population.

17. The method of claim 16, further comprising repeating the steps of claim 16 with a filter membrane comprising a different size pore than the filter membrane of claim 16.

18. The method of claim 16, further comprising allowing the nematodes to settle to a bottom of the first sample vessel, second sample vessel, or both.

19. The method of claim 18, wherein allowing the nematodes to settle to the bottom of the sample vessel comprises subjecting the sample vessel to a rotating force.

20. The method of claim 16, wherein step b) further comprises using absorbent material to aid drawing the nematodes through the filter membrane.
